(19) United States
(12) Patent Application Publication
(10) Pub. No.: US 2013/0330729 A9
(43) Pub. Date: Dec. 12, 2013

(54) INHIBITOR LEACHING RESISTANT NUCLEIC ACID STORAGE REAGENT

(71) Applicant: Matt Ewert, St. Petersburg, FL (US)

(72) Inventor: Matt Ewert, St. Petersburg, FL (US)

(73) Assignee: Imigene, Inc., Saint Petersburg, FL (US)

(21) Appl. No.: 13/655,238

(22) Filed: Oct. 18, 2012

Related U.S. Application Data
(60) Provisional application No. 61/549,831, filed on Oct. 21, 2011.

(51) Int. Cl. C12Q 1/68

(52) U.S. Cl. CPC C12Q 1/6806
USPC 435/6.12; 435/91.2; 435/287.2

(57) ABSTRACT

The invention provides compositions and methods related to a nucleic acid storage reagent.

### RELATED APPLICATIONS

This application claims the benefit under 35 USC 119(e) of the filing date of U.S. provisional patent application 61/549,831, filed Oct. 21, 2011 entitled “INHIBITOR LEACHING RESISTANT NUCLEIC ACID STORAGE REAGENT”. The entire teachings and contents of the referenced provisional application are incorporated herein by reference.

### BACKGROUND OF THE INVENTION

Proper nucleic acid storage is essential for biological research and industry. Commercially available nucleic acid storage reagents often include Tris, a well-established buffer, and EDTA, an efficient chelator of undesirable divalent cations. Nucleic acid storage reagents often contain contaminating nucleic acids, such as bacterial DNA, which may interfere with diagnostic or research applications. In order to remove these contaminating nucleic acids, a storage buffer may be exposed to an ultraviolet light in order to cross-link the contaminating nucleic acids such that they will not interfere with downstream reactions, e.g., PCR. As described herein, it has been discovered that storing nucleic acids in UV-treated Tris buffer in plastic containers can inhibit downstream reactions utilizing the stored nucleic acids, such as PCR. Thus, a need exists for nucleic acid storage reagents that can undergo UV treatment without compromising further use of the nucleic acid stored in the reagent.

### SUMMARY OF THE INVENTION

As described herein, the inventors discovered, unexpectedly, that storage of nucleic acids in UV-treated Tris-containing buffers that additionally contain EDTA and Sodium Azide results in reduced inhibitory effects on downstream analysis of the stored nucleic acid (e.g., PCR analysis). Accordingly, aspects of the invention relate to compositions comprising Tris, EDTA, and Sodium Azide, methods of producing said compositions, containers comprising said compositions and methods of use of said compositions.

In one aspect, the invention relates to a composition, comprising Tris, ethylenediaminetetraacetic acid (EDTA), and sodium azide (NaN₃), wherein the composition has been previously exposed to an artificial source of ultraviolet irradiation. In some embodiments, the Tris is a Tris acid and/or a Tris base. In some embodiments, the Tris is a Tris acid. In some embodiments, the Tris is a Tris base. In some embodiments, the composition is a liquid. In some embodiments, the composition is dry. In some embodiments, the composition is lyophilized. In some embodiments, the composition is a solution and wherein Tris is present in an amount of 1.0 mM to 50 mM, EDTA is present in an amount of 0.05 mM to 2.0 mM, and NaN₃ is present in an amount of 0.01 to 0.3%. In some embodiments, the exposure to ultraviolet irradiation is for at least 15 minutes. In some embodiments, the exposure to ultraviolet irradiation is for 1 to 4 hours. In some embodiments, the ultraviolet irradiation is at a wavelength range from 250 to 320 nm. In some embodiments, the ultraviolet irradiation is at UV energy dosage range from 60 to 200 mJ/cm².

In another aspect, the invention related to a container, comprising (a) a composition comprising Tris, EDTA, and NaN₃, wherein the composition has been previously exposed to an artificial source of ultraviolet irradiation and (b) a nucleic acid. In some embodiments, (a) and (b) are frozen. In some embodiments, (a) and (b) are in a solution and wherein Tris is present in an amount of 1.0 mM to 50 mM, EDTA is present in an amount of 0.05 mM to 2.0 mM, and NaN₃ is present in an amount of 0.01 to 0.3%. In some embodiments, the container is plastic. In some embodiments, the plastic comprises a polypropylene, a polystyrene, a polycarbonate, a cyclo-olefin, or a mixture thereof. In some embodiments, the nucleic acid has not been previously exposed to an artificial source of ultraviolet irradiation. In some embodiments, the nucleic acid is a DNA or cDNA. In some embodiments, the exposure to ultraviolet irradiation is for at least 15 minutes. In some embodiments, the exposure to ultraviolet irradiation is for 1 to 4 hours. In some embodiments, the ultraviolet irradiation is at a wavelength range from 250 to 320 nm. In some embodiments, the ultraviolet irradiation is at UV energy dosage range from 60 to 200 mJ/cm².

Aspects of the invention also relate to a method of producing a reagent, the method comprising:
(a) combining Tris, EDTA, and NaN₃ in a solution; and
(b) exposing the solution to an artificial source of ultraviolet irradiation, thereby producing a reagent.
   In some embodiments, the method further comprises drying the reagent. In some embodiments, the method further comprises lyophilizing the reagent. In some embodiments, Tris is present in an amount of 1.0 mM to 50 mM, EDTA is present in an amount of 0.05 mM to 2.0 mM, and NaN₃ is present in an amount of 0.01 to 0.3%. In some embodiments, the exposure to ultraviolet irradiation is for at least 15 minutes. In some embodiments, the exposure to ultraviolet irradiation is for 1 to 4 hours. In some embodiments, the ultraviolet irradiation is at a wavelength range from 250 to 320 nm. In some embodiments, the ultraviolet irradiation is at UV energy dosage range from 60 to 200 mJ/cm². In some embodiments the reagent is in a plastic container when it is exposed to ultraviolet radiation.

Other aspects of the invention relate to a product obtainable by any of the methods described above and herein.

A further aspect of the invention relates to methods of use of the compositions described herein. In some embodiments, the method comprises performing a polymerase chain reaction (PCR) in the presence of a composition comprising Tris, EDTA, and sodium azide, wherein the composition has been previously exposed to an artificial source of ultraviolet irradiation. In some embodiments, the PCR is real-time PCR. In some embodiments, Tris is present in an amount of 1.0 mM to 50 mM, EDTA is present in an amount of 0.05 mM to 2.0 mM, and NaN₃ is present in an amount of 0.01 to 0.3%. In some embodiments, the exposure to ultraviolet irradiation is for at least 15 minutes. In some embodiments, the exposure to ultraviolet irradiation is for 1 to 4 hours. In some embodiments, the ultraviolet irradiation is at a wavelength range from 250 to 320 nm. In some embodiments, the ultraviolet irradiation is at UV energy dosage range from 60 to 200 mJ/cm².

It is to be understood by one of skill in the art that compounds closely related to EDTA and sodium azide such as, e.g., EGTA or aryl azide, may be substituted for EDTA and/or sodium azide, and it is believed by the inventor that substitution will result in advantages similar to those obtained in the present invention.

### BRIEF DESCRIPTION OF THE DRAWINGS

FIG. 1A is a graph showing PCR efficiency of UV-treated Tris-buffered DNA stored in SSI 1.5 mL tubes over 13 days. FIG. 1B is a graph showing PCR efficiency of UV-treated Tris EDTA buffered DNA stored in SSI 1.5 mL tubes over 13 days. FIG. 1C is a graph showing PCR efficiency of Tris-buffered DNA stored in SSI 1.5 mL tubes over 13 days.

FIG. 2A is a graph of PCR efficiency of UV-treated Tris, EDTA, NaN₃-buffered (UVATE) DNA stored in SSI 1.5 mL tubes over 13 days. FIG. 2B is a graph of PCR efficiency of Tris, EDTA, NaN₃-buffered (ATE) DNA stored in SSI 1.5 mL tubes over 13 days.

FIG. 3A is a graph of PCR efficiency of UV-treated Tris EDTA-buffered DNA stored in SSI 1.5 mL tubes over 13 days. FIG. 3B is a graph of PCR efficiency of UV-treated Tris NaN₃ buffered DNA stored in SSI 1.5 mL tubes over 13 days. FIG. 3C is a graph of PCR efficiency of UV-treated Tris, EDTA, NaN₃-buffered (UVATE) DNA stored in SSI 1.5 mL tubes over 13 days.

FIG. 4A is a graph showing the effect of temperature on Ct for DNA after 14 days storage in UVATE buffer in different plasticware. FIG. 4B is a graph showing the effect of temperature on Ct for DNA after 14 days storage in ATE buffer in different plasticware.

FIG. 5A is a graph of PCR efficiency of DNA stored in ATE buffer within domed top PCR tubes over 14 days. FIG. 5B is a graph of PCR efficiency of DNA stored in UVATE buffer within domed top PCR tubes over 14 days.

FIG. 6A is a graph of PCR efficiency of DNA stored in ATE buffer within SSI 1.5 mL tubes over 14 days. FIG. 6B is a graph of PCR efficiency of DNA stored in UVATE buffer within SSI 1.5 mL tubes over 14 days.

FIG. 7A is a graph of PCR efficiency of DNA stored in ATE buffer within Sarstedt 2 ml tubes over 14 days. FIG. 7B is a graph of PCR efficiency of DNA stored in UVATE buffer within Sarstedt 2 ml tubes over 14 days.

FIG. 8A is a graph of PCR efficiency of DNA stored in ATE buffer within Sarstedt 1.5 ml tubes over 14 days. FIG. 8B is a graph of PCR efficiency of DNA stored in UVATE buffer within Sarstedt 1.5 ml tubes over 14 days.

FIG. 9A is a graph of PCR efficiency of DNA stored in ATE buffer within Falcon 15 ml tubes over 14 days. FIG. 9B is a graph of PCR efficiency of DNA stored in UVATE buffer within Falcon 15 ml tubes over 14 days.

FIG. 10A is a graph of PCR efficiency of DNA stored in ATE buffer within Sarstedt 50 ml tubes over 14 days. FIG. 10B is a graph of PCR efficiency of DNA stored in UVATE buffer within Sarstedt 50 ml tubes over 14 days.

FIG. 11A is a graph showing the effect of temperature on Ct for DNA after 14 days storage in UVATE buffer in different plasticware. FIG. 11B is a graph showing the effect of temperature on Ct for DNA after 14 days storage in ATE buffer in different plasticware.

FIG. 12A is a graph of PCR efficiency of ATE-buffered DNA stored in SSI 1.5 mL tubes over 13 days. FIG. 12B is a graph of PCR efficiency of ATE-buffered DNA stored in Sarstedt 1.5 mL tubes over 13 days.

FIG. 13A is a graph of PCR efficiency of UVATE-buffered DNA stored in SSI 1.5 mL tubes over 13 days. FIG. 13B is a graph of PCR efficiency of UVATE-buffered DNA stored in Sarstedt 1.5 mL tubes over 13 days.

FIG. 14A is a graph of PCR efficiency of Tris-buffered DNA stored in SSI 1.5 mL tubes over 13 days. FIG. 14B is a graph of PCR efficiency of Tris-buffered DNA stored in Sarstedt 1.5 mL tubes over 13 days.

FIG. 15A is a graph of PCR efficiency of UV Tris-buffered DNA stored in SSI 1.5 mL tubes over 13 days. FIG. 15B is a graph of PCR efficiency of UV Tris-buffered DNA stored in Sarstedt 1.5 mL tubes over 13 days.

FIG. 16A is a graph of PCR efficiency of UV Tris-EDTA-buffered DNA stored in SSI 1.5 mL tubes over 13 days. FIG. 16B is a graph of PCR efficiency of UV Tris-EDTA-buffered DNA stored in Sarstedt 1.5 mL tubes over 13 days.

FIG. 17A is a graph of PCR efficiency of UV Tris-NaN3-buffered DNA stored in SSI 1.5 mL tubes over 13 days. FIG. 17B is a graph of PCR efficiency of UV Tris-NaN3-buffered DNA stored in Sarstedt 1.5 mL tubes over 13 days.

FIG. 18A is a graph of PCR efficiency of H20-buffered DNA stored in SSI 1.5 mL tubes over 13 days. FIG. 18B is a graph of PCR efficiency of H20-buffered DNA stored in Sarstedt 1.5 mL tubes over 13 days.

FIG. 19A is a graph of PCR efficiency of UVH20-buffered DNA stored in SSI 1.5 mL tubes over 13 days. FIG. 19B is a graph of PCR efficiency of UVH20-buffered DNA stored in Sarstedt 1.5 mL tubes over 13 days.

FIG. 20A is a graph showing the effect of temperature on Cₜ for DNA after 13 days storage in different buffers in Sarstedt 1.5 mL tubes. FIG. 20B is a graph showing the effect of temperature on Ct for DNA after 13 days storage in different buffers in SSI 1.5 mL tubes.

### DETAILED DESCRIPTION OF THE INVENTION

Nucleic acids, especially bacterial and fungal nucleic acids, can be found on laboratory surfaces and in the air. As a result, these nucleic acids can easily contaminate an open container or a reagent in a container. These contaminating nucleic acids can interfere with detection of target nucleic acids, especially when the target nucleic acids are present in very low amounts. This is especially the case in applications such as ancient DNA research, forensic analyses, wildlife studies and ultrasensitive diagnostics, as the target nucleic acid which is to be used as a template for amplification may be present in quantities that are even less than the contaminating nucleic acid.

In order to avoid amplification of such contaminating nucleic, buffers and their containers are pre-treated with UV light to cross-link the contaminating nucleic acids. This cross-linking prevents the contaminating nucleic acids from interfering with downstream analysis of target nucleic acids, by preventing amplification of the contaminating DNA. For example, UV treatment of contaminating DNA causes the formation of pyrimidine-pyrimidine photoadducts that interfere with the ability of the contaminating DNA to act as a template for amplification.

Tris is a common component found in most nucleic acid storage reagents. As described herein, it has been discovered, unexpectedly, that UV treatment of a Tris buffer in a plastic container resulted in reduced PCR efficiency versus the PCR efficiency when using a Tris buffer in a plastic container that had not been UV treated. Without wishing to be bound by any theory of the invention, it is known that leachates from plastic vessels inhibit PCR. It is believed by the inventor that UV-treated Tris-containing reagents in a plastic vessel either contribute to a higher level of leachates and/or a higher activity of such leachates, thereby negatively affecting PCR reactions.

Surprisingly, addition of EDTA and sodium azide to the Tris-containing reagent (the combination of Azide, Tris and EDTA referred to herein as ATE) before UV-treatment essentially abolishes the PCR inhibition caused by UV-treated Tris-containing reagents contained in a plastic vessel. This is especially surprising, as use of an ATE solution in the absence of UV treatment also results in PCR inhibition similar to that caused by UV-treated Tris-containing reagents. Thus, unexpectedly, if one wants to gain the full benefits of a UV-treated Tris-containing storage and/or reaction buffer, the buffer desirably contains EDTA and sodium azide before UV-treatment. Without being bound by any theory of the invention, the inventor believes that the UV treatment physically alters the ATE in solution, producing a novel composition of matter. The inventor has discovered that use of UV treated ATE (UVATE) aids PCR reactions by preventing adverse effects on PCR reactions resulting from use of plastic storage and/or plastic reaction vessels, Tris and UV treatment. Accordingly aspects of the invention related to compositions comprising Tris, EDTA, and sodium azide that have been treated with ultraviolet irradiation. Other aspects of the invention related to methods of making said compositions and of use of said compositions.

### Compositions

Some aspects of the invention relate to compositions comprising Tris, ethylenediaminetetraacetic acid (EDTA), and sodium azide (NaN3), wherein the composition has been previously exposed to an artificial source of ultraviolet irradiation.

Tris is an organic compound used extensively in biological research and in industry as a component of buffer solutions. Tris occurs as both as an acid and as a base. The basic form Tris(hydroxymethyl)aminomethane (also referred to as Tris base, Trizma™, Trisamine, THAM, Tromethamine, Trometamol, Tromethane) has the formula NH₂C(CH₂OH)₃. The acidic form of Tris (also referred to as Tris-HCl, Tris Hydrochloride or Trizma™ hydrochloride) has the formula NH₂C(CH₂OH)₃.HCl. The use of Tris in biological research occurs in part because the useful buffer range for Tris is pH 7-9, which coincides with the physiological pH of living organisms. Accordingly, in some embodiments, compositions of the invention have a pH of about 7-9. In some embodiments, compositions of the invention have a pH of about 6-10. It is to be understood that the compositions described herein can include any form of Tris.

Ethylenediaminetetraacetic acid (most often referred to as EDTA, but also called Diaminoethane-tetraacetic acid, Edetic acid, and Ethylenedinitrilo-tetraacetic acid) is a chelating agent that is available from a variety of commercial vendors. EDTA is probably the most widely recognized member of a family of related chemical compounds known as polyaminocarboxylic acids. It is commonly used in biological research for its ability to sequester divalent cations, such as Ca²⁺. EDTA can be produced as a salt, e.g., disodium EDTA, dipotassium EDTA, and calcium disodium EDTA. It is to be understood that the compositions described herein can include EDTA or a salt thereof. Like EDTA, related polyaminocarboxylic acids are chelating agents characterized by multiple carboxylic acid moieties and multiple amine groups. Substitution of EDTA with another polyaminocarboxylic acid is contemplated herein. In addition to EDTA, polyaminocarboxylic acids include, without limitation, ethylene glycol-bis(2-aminoethyl)-N,N,N′,N′-tetraacetic acid (EGTA), diethylenetriaminepentaacetic acid (DTPA), hydroxyethylethylenediaminetriacetic acid (HEDTA), methylethylenediaminetriacetic acid (MEDTA), diaminocyclohexanetetraacetic acid (DCTA), 1,2-bis(o-aminophenoxy)ethane-N,N,N′,N′-tetraacetic acid (BAPTA), 1,3-propylenediaminetetraacetic acid (PDTA), trimethylenediaminetetraacetic acid (TMDTA), tetramethylenediaminetetraacetic acid (TMEDTA), and nitrilotriacetic acid (NTA). In some embodiments, EDTA is replaced with EGTA. In some embodiments, both EDTA and EGTA are present.

Sodium azide (NaN₃, also referred to as NaAzide, sodium trinitride, smite, and azium) is a highly toxic inorganic substance with applications in biochemistry and medicine. Sodium azide is capable of acting as a bacteriostatic by inhibiting cytochrome oxidase in gram-negative bacteria. Because of this activity, sodium azide is often used in hospitals and laboratories as a biocide. It is to be understood that the compositions described herein can include NaN₃ or any related azide compound appropriate for nucleic acid storage or PCR, e.g., an aryl azide or an acyl azide.

Any UV exposure or artificial source of UV irradiation described herein can be used. In some embodiments, the exposure to ultraviolet irradiation is for at least 15 minutes. In some embodiments, the exposure to ultraviolet irradiation is for 1 to 4 hours. In some embodiments, the ultraviolet irradiation wavelength range is from 250 to 320 nm. In some embodiments, the UV energy dosage range is from 60 to 200 mJ/cm².

Compositions of the invention can be in any state of matter, e.g., liquid, dry solid or frozen. In some embodiments, compositions of the invention can be a liquid. In some embodiments, compositions of the invention are frozen. In some embodiments, compositions of the invention are dry, e.g., a powder. In some embodiments, compositions of the invention are lyophilized (also sometimes referred to as freeze-drying). Methods of drying, lyophilizing and powdering are well-known in the art (see, e.g., Day and Stacey (2007). Cryopreservation and Freeze-Drying Protocols (Methods in Molecular Biology). Humana Press. 2nd edition and Kennedy, John F. and Joaquim M. S. Cabral (1993). Recovery Processes for Biological Materials. John Wiley & Sons Ltd.).

In some embodiments, the composition is in a solution or is a solution. In some embodiments, Tris is present in the solution in an amount of 0.1 mM to 100 M, 0.1 mM to 50 M, 0.5 mM to 1M, 1 mM to 1M, 1 mM to 1M, 1 mM to 500 mM, 1 mM to 100 mM, or 1 mM to 50 mM. In some embodiments, Tris is present in an amount of 1.0 mM to 50 mM. In some embodiments, Tris is present in the solution in an amount of 10 mM. In some embodiments, EDTA is present the solution in an amount of 0.01 mM to 100 mM, 0.01 mM to 10 mM, 0.05 mM to 5 mM, or 0.05 mM to 2.0 mM. In some embodiments, EDTA is present in the solution in an amount of 0.05 mM to 2.0 mM. In some embodiments, EDTA is present in the solution in an amount of 0.1 mM. In some embodiments, NaN₃ is present in the solution in an amount of 0.001 to 10%, 0.05 to 5%, 0.01 to 1%, 0.01 to 0.5%, or 0.01 to 0.3%. In some embodiments, NaN₃ is present in the solution in an amount of 0.01 to 0.3%. In some embodiments, Tris is present in the solution in an amount of 1.0 mM to 50 mM, EDTA is present in an amount of 0.05 mM-2.0 mM, and NaN₃ is present in an amount of 0.01 to 0.3%. It is desirable to produce compositions that can serve as a stock from which aliquots can be made, and thus it is to be appreciated that compositions of the invention can be in a more concentrated form (e.g., 10×, 100×, 1000× of the ranges recited herein) that is diluted before use.

The compositions of the invention have many uses, e.g., as a buffer for nucleic acid storage or for PCR. As such, in some instances it is desirable to include additional components common to nucleic acid storage buffers and PCR reagents. In some embodiments, the composition further comprises sodium chloride. In some embodiments, the composition further comprises magnesium chloride. In some embodiments, the composition further comprises calcium chloride. In some embodiments, the composition further comprises at least one monovalent cation (e.g., sodium or potassium ions). In some embodiments, the composition further comprises at least one divalent cation (e.g., magnesium or manganese ions). In some embodiments, the composition further comprises a nucleic acid primer. In some embodiments, the composition further comprises a polymerase. In some embodiments, the composition further comprises a deoxynucleoside triphosphate (dNTP).

### Containers

In another aspect, the invention provides a container, comprising: a composition described herein and a nucleic acid. In some embodiments, the container comprises: (a) a composition comprising Tris, EDTA, and NaN3, wherein the composition has been previously exposed to an artificial source of ultraviolet irradiation; and (b) a nucleic acid. In some embodiments, the container is a nucleic acid storage container. In some embodiments, the container is a PCR container for conducting a PCR reaction. Containers for storing nucleic acids and/or PCR containers are commercially available and well-known in the art. Examples of containers for nucleic acid storage and/or PCR containers include those made by companies such as BD Biosciences (Franklin Lakes, N.J., makers of Falcon® tubes), Scientific Specialties Inc. (SSI, Lodi, Calif.), Eppendorf (Hamburg, Germany), and Sarstedt (Numbrecht, Germany). The container may be made out of any material suitable for nucleic acid storage and/or PCR (e.g., glass or plastic). In a preferred embodiment, the container is plastic. The plastic can comprise any polymer suitable for nucleic acid storage and/or PCR, e.g., a polypropylene, a polystyrene, a polycarbonate, a cyclo-olefin, or a mixture thereof. In some embodiments, the container and/or the plastic comprises leachates. Examples of leachates include, but are not limited to, releasing agents and biocides. In the context of plastic manufacture, release agents (also known as de-molding agents, form oils, parting agents or form releaser) are substances used in molding and casting that aid in the separation of a mold from the material being molded and reduce imperfections in the molded surface. Examples of releasing agents include, but are not limited to, erucamide, steramide, bisamide, ethylene bisstearamide, glyceryl monostearate, oleamide, stearyl stearamide, and stearyl erucamide. Biocides are compounds that prevent growth of contaminants, e.g., bacteria and fungi. Examples of biocides include, but are not limited to quaternary ammonium compounds, cyclodextrins, formaldehyde derivatives, halogen compounds, heterocyclics with anionic groups, iodophors, N-halamine, nitro compounds, organometallics, phenolic compounds, polychloro phenoxy phenol, and polyhexamethylene biguanide.

The container can be any shape and size. In some embodiments, the container is a tube. The tube may be cylindrical, conical, rectangular, triangular, or have an irregular shape (e.g., the shape of a 1.5 mL Eppendorf tube or 15 mL Falcon tube). The tube may optionally have a cap. Suitable caps include, but are not limited to, snap-on caps, dome-top caps and screw caps. Suitable container sizes by volume include, but are not limited to, 0.1 mL, 0.2 mL, 0.5 mL, 1.5 mL, 2.0 mL, 5 mL, 15 mL, and 50 mL, 100 mL, 500 mL, 1 L, 5 L, 10 L, or 100 L. In some embodiments, the container is a plate. Examples of plates include microwell plates (also sometimes referred to as microtiter plates or microplates, see, e.g., Nunc microwell plates from Thermo Scientific, Waltham, Mass.). The plate may have several wells, e.g., 6, 12, 24, 48, 96, 192, 384, 769, 1536 or more wells.

In some embodiments, the container and its contents are maintained at a specific temperature or within a range of temperatures (e.g., −80 degrees Celsius to 0 degrees Celsius, 0 degrees Celsius to 18 degrees Celsius, 18 degrees Celsius to 28 degrees Celsius, or 28 degrees Celsius to 37 degrees Celsius). In some embodiments, the container and its contents are maintained at a specific temperature or within a range of temperatures for a minimum amount of time, e.g., at least 1 hour, 2 hours, 3 hours, 4 hours, 5 hours, 10 hours, 24 hours, 36 hours, 48 hours, 3 days, 4 days, 5 days, 6 days, 7 days, 10 days, 14 days, 3 weeks, 4 weeks, 2 months, 3 months, 6 months, 12 months or longer. By way of example, the container could be stored in a refrigerator or a freezer until the nucleic acid in the container is needed to run a reaction, e.g. PCR. Accordingly, in some embodiments, the contents of the container are frozen (i.e., in a frozen state or solid). In other embodiments, the contents of the container are liquid. The nucleic acid contained within the container can be any type of nucleic acid, e.g., an RNA, a DNA, or a cDNA. Examples of DNA include, but are not limited to, a plasmid, a vector, a primer, a probe, fragments of DNA (e.g., generated by PCR or a restriction enzyme reaction), viral DNA, or genomic DNA, or combinations thereof. Examples of RNA include, but are not limited to, mRNA, non-coding RNAs, viral RNA, tRNAs, or combinations thereof. In some embodiments, the nucleic acid is a DNA. In some embodiments, the nucleic acid is a cDNA or a DNA.

### UV Irradiation

In some embodiments, compositions and methods of the invention involve UV treatment by exposure to ultraviolet (UV) irradiation (also referred to herein as UV light). In some embodiments, compositions of the invention are exposed to an artificial source of UV irradiation. In some embodiments, methods comprise a step of exposure to an artificial source of UV irradiation. As defined herein, exposure to an artificial source of UV irradiation means passing UV light from an artificial source through a composition of matter or shining a UV light from an artificial source on a composition of matter.

UV occurs in the range 10 nm to 400 nm, corresponding to photon energies from 3 eV to 124 eV. In some embodiments, an artificial source of ultraviolet irradiation or light is provided. Any artificial source of UV irradiation or light is contemplated. Examples of artificial sources of UV light include, but are not limited, a black light (a lamp that emits long-wave UV radiation, typically containing europium-doped strontium fluoroborate (SrB4O7F:Eu2+), europium-doped strontium borate (SrB4O7:Eu2+), or lead-doped barium silicate (BaSi2O5:Pb+)), a short wave ultraviolet lamp, a gas-discharge lamp, a UV light-emitting diode (LED), or a UV laser. In some embodiments, the artificial source of UV irradiation is a 254 nm lamp (e.g., an XL-1500 UV Crosslinker, Spectronics, Westbury, N.Y.).

The artificial source of UV irradiation may emit all wavelengths of UV light or a subset of wavelengths of UV light. Accordingly, UV irradiation as described herein may be at a particular wavelength or range of wavelengths, e.g., 10 to 400 nm, 100 to 400 nm, 200 to 400 nm, 200 to 350 nm, or 250 to 320 nm. In some embodiments, the UV irradiation wavelength range is from 250 to 320 nm. In some embodiments, the UV irradiation is at 254 nm.

In some embodiments, the exposure to ultraviolet irradiation is for a defined period of time. The exposure time will depend on several factors, e.g., the volume of the container holding a composition and the relative depth to the UV source. The exposure time will also depend on whether contaminating DNA is present and whether cross-linking of the contaminating DNA is desired. For example, a 1 L volume of water that presents a 7.5 cm depth relative to the UV source would require approximately 4 hours of UV exposure to properly cross-link contaminating DNA (see Example 1). Thus, a smaller vessel may require less UV exposure time and a larger vessel may require more UV exposure time. Accordingly, in some embodiments, the exposure to ultraviolet irradiation is for at least 15 minutes. In some embodiments, the exposure to ultraviolet irradiation is for at least 1 hour. In some embodiments, the exposure to ultraviolet irradiation is for 1 to 4 hours.

In some embodiments, the exposure to UV irradiation is at a defined energy dosage range. In some embodiments, the UV energy dosage range is from, e.g., 10 to 500 mJ/cm², 50 to 500 mJ/cm², 50 to 400 mJ/cm², 50 to 300 mJ/cm², 50 to 200 mJ/cm², 60 to 200 mJ/cm², 100 to 200 mJ/cm², 100 to 150 mJ/cm², or 100 to 125 mJ/cm². In some embodiments, the UV energy dosage range is from 60 to 200 mJ/cm². In some embodiments, the UV energy dosage is 120 mJ/cm².

### Methods of Manufacture and Products Obtainable by Said Methods

In one aspect, the invention provides methods of producing the compositions described herein. In another aspect, the invention relates to a product obtainable by the methods of producing described herein. In some embodiments, the method comprises:
(a) combining Tris, EDTA, and sodium azide in a solution; and
(b) exposing the solution to an artificial source of ultraviolet irradiation, thereby producing a reagent.
   In some embodiments, the method further comprises drying the reagent. In some embodiments, the method further comprises lyophilizing the reagent. Methods of drying and lyophilizing are well-known in the art (see, e.g., Day and Stacey (2007). Cryopreservation and Freeze-Drying Protocols (Methods in Molecular Biology). Humana Press. 2nd edition and Kennedy, John F. and Joaquim M. S. Cabral (1993). Recovery Processes for Biological Materials. John Wiley & Sons Ltd.).

Any UV exposure or artificial source of UV irradiation described herein can be used in a method of producing the compositions described herein. In some embodiments, the exposure to ultraviolet irradiation is for at least 15 minutes. In some embodiments, the exposure to ultraviolet irradiation is for 1 to 4 hours. In some embodiments, the ultraviolet irradiation wavelength range is from 250 to 320 nm. In some embodiments, the UV energy dosage range is from 60 to 200 mJ/cm². In some embodiments, Tris is present in an amount of 1.0 mM to 50 mM, EDTA is present in an amount of 0.05 mM to 2.0 mM, and NaN3 is present in an amount of 0.01 to 0.3%. In some embodiments, the solution further comprises sodium chloride.

In some embodiments, the reagent is produced in a glass container. In some embodiments, the reagent is produced in a plastic container. In some embodiments, the reagent is in a container that also contains contaminating nucleic acid and the reagent is produced in the presence of, and at the same time as, cross-linking the contaminating nucleic acid.

### Methods of Use

In another aspect of the invention, methods of use of the compositions described herein are provided. In some embodiments, the compositions described herein are used during analysis of a nucleic acid. In some embodiments, the compositions described herein are used during a polymerase chain reaction. In some embodiments, the method comprises performing a polymerase chain reaction (PCR) in the presence of a composition comprising Tris, EDTA, and sodium azide, wherein the composition has been previously exposed to an artificial source of ultraviolet irradiation. In some embodiments, the method comprises adding UVATE to a nucleic acid before running PCR. Polymerase chain reactions are well-known in the art (see, e.g. Park. 2011. PCR Protocols (Methods in Molecular Biology). Humana Press; 3rd edition. Vol. 687 and King. 2010. RT-PCR Protocols: Second Edition (Methods in Molecular Biology). Humana Press; 2nd edition.). Any type of PCR is contemplated. Exemplary components used during a PCR reaction include, but are not limited to, a nucleic acid template to be amplified, primers, a polymerase, deoxynucleoside triphosphates (dNTPs), buffer solution, divalent cations, (e.g., magnesium or manganese ions), and monovalent cations (e.g., potassium ions). Examples of PCR reactions include, but are not limited to: Amplified fragment length polymorphism PCR, Allele-specific PCR, Alu PCR, Assembly PCR, Asymmetric PCR, Colony PCR, Dial-out PCR, Helicase dependent amplification, Hot start PCR, Inverse PCR, In-situ PCR, Inter-simple sequence repeat PCR, Ligation-mediated PCR, Methylation-specific PCR, Miniprimer PCR, Multiplex Ligation-dependent Probe Amplification, Multiplex-PCR, Nested PCR, Overlap-extension PCR, Solid Phase PCR, Reverse transcriptase PCR, Touchdown PCR and Real time PCR (sometimes also called quantitative PCR). In some embodiments, the PCR is real-time PCR.

In some embodiments, the compositions described herein are used during purification, elution, or extraction of a nucleic acid from a sample. Methods of nucleic acid purification, elution, and extraction are well-known in the art (see, e.g., Walker. (2012) Methods in Molecular Biology. Humana Press; Rapley. (2000) The Nucleic Acid Protocols Handbook. Springer; and Farrell. (2009) RNA Methodologies: A Laboratory for Isolation and Characterization. Academic Press). In some embodiments, the method comprises extracting a nucleic acid in the presence of a composition described herein. In some embodiments, the method comprises providing a biological sample that includes a nucleic acid, binding the nucleic acid to a matrix, whereby a bound nucleic acid is produced, and contacting the bound nucleic acid with a composition described herein, whereby the nucleic acid is extracted. In some embodiments, the method comprises contacting a nucleic acid bound to a matrix with a composition of the invention. Any matrix capable of binding a nucleic acid is contemplated. In some embodiments, the matrix is glass.

In some embodiments the method is one for preparing a storage buffer, where potentially contaminating nucleic acid in the buffer cross-linked. The invention also involves the use of such a storage buffer for storing a target nucleic acid, which target nucleic acid can be used at a later time for analysis, such as in PCR.

The invention will be more fully understood by reference to the following examples. These examples, however, are merely intended to illustrate the embodiments of the invention and are not to be construed to limit the scope of the invention.

### EXAMPLES

### Example 1

### Preparation of Cross-Linked DNA in a Tris, EDTA, and Sodium Azide Composition

Contaminating DNA found in buffers can negatively influence sample analysis, e.g. PCR analysis. A 10 mM Tris, 0.1 mM EDTA, and 0.04% Sodium Azide solution was prepared in a 1 L container that was 7.5 cm deep and intentionally contaminated with E. coli DNA. The solution was UV treated with 120 mJ/cm² at 254 nm (Spectronics XL-1500 UV Crosslinker, Westbury, N.Y.) for 1, 2, 3 and 4 hours. Contaminating E. coli DNA was detected using PCR. It was found that UV treatment for 4 hours was necessary to effectively cross-link the contaminating DNA such that it was not detectable by PCR.

### Example 2

### Comparison of PCR Kinetics of Tris, UV-Treated Tris, UV-Treated Tris EDTA, UV-Treated Tris NaAzide, ATE, and UV-Treated ATE Buffers

### Materials and Methods

Different buffers were contained within different types of plastic to determine whether they had any impact on cycle threshold (CO value of S. pneumonia or E. coli DNA PCR. 2 microliters of S. pneumonia or E. Coli DNA was seeded into 2 mL of:

1) a 10 mM Tris buffer

2) a UV-treated 10 mM Tris buffer

3) a UV-treated 10 mM Tris and 0.1 mM EDTA buffer

4) a UV-treated 10 mM Tris and 0.04% Sodium Azide buffer

5) a 10 mM Tris, 0.1 mM EDTA, and 0.04% Sodium Azide (ATE) buffer; or

6) a UV-treated ATE buffer (UVATE),

to a final concentration of 0.01 ng/microliter of DNA. UV treatment of the buffers was with 120 mJ/cm² at 254 nm (Spectronics XL-1500 UV Crosslinker, Westbury, N.Y.) for up to 4 hours. An aliquot of each fresh solution was amplified immediately to obtain a baseline Cₜ value. 50 microliter aliquots were dispensed into 1.5 mL Scientific Specialties Inc. (SSI, Lodi, Calif.) tubes and stored for 1, 6, and 13 days at −20 degrees Celsius, 4 degrees Celsius, 25 degrees Celsius (room temperature), and 37 degrees Celsius. At each time point, 50 microliters from each buffer was removed and amplified using real-time PCR.

For real-time PCR analysis, a single mastermix was assembled according to the following concentrations: 0.5 (S. pneumoniae) or 0.625 (E. coli) unites per reaction of PLATINUM Taq polymerase (Invitrogen, Carlsbad, Calif.), 1×PCR buffer (Invitrogen), 0.2 mM for each dNTP (Invitrogen), 4.5 mM (S. pneumoniae) or 5.5 mM (E. coli) MgCl₂ (Invitrogen), 0.01% v/v bovine serum albumin (Sigma, St. Louis, Mo.) and 6.5 micromolar propidium monoazide (PMA, Biotium, Hayward, Calif.). Contaminating DNA in the PMA was cross-linked by exposure to a light source for a total of 20 min, in alternative light/dark cycles of 30 seconds and 60 seconds respectively. After PMA treatment, 0.2 micromolar of each of the forward/reverse primer and FAM-labeled probe was added. The mastermix was stored in 200 microliter aliquots at −80 degrees Celsius until required, ensuring every time point had an identical number of freeze/thaw cycles. At each time point, mastermix was dispensed into a Genedisc-72 ring using a liquid handling robot (QIAgility, Qiagen, Hilden, Germany). 10 microliters of each of the DNA samples, positive control (E. coli) and negative control (buffer alone) were inoculated in triplicate by hand into the Genedisc-72 ring, giving a final volume of 25 microliters per reaction (10 microliter sample and 15 microliters mastermix). The ring was sealed using thermal sealing film. Amplification was performed on a Rotor-Gene Q Thermal Cycler (Qiagen, Hilden, Germany) using the following reaction profile: 94 degrees Celsius for 2 min, followed by 50 cycles of: 94 degrees Celsius for 15 sec, 60 degrees for 30 sec, and 72 degrees for 20 sec. Gains were set automatically before the first fluorescence acquisition using the default settings. Fluorescence acquisition to the green channel was performed after each extension. Analysis was performed using a cycle threshold (Cₜ)=0.05 and Slope Correction=ON.

### Results

Real-time PCR results were compared for S. pneumonia or E. Coli DNA seeded into:

1) a 10 mM Tris buffer

2) a UV-treated 10 mM Tris buffer

3) a UV-treated 10 mM Tris and 0.1 mM EDTA buffer

4) a UV-treated 10 mM Tris and 0.04% Sodium Azide buffer

5) a 10 mM Tris, 0.1 mM EDTA, and 0.04% Sodium Azide (ATE) buffer; or

6) a UV-treated ATE buffer (UVATE),

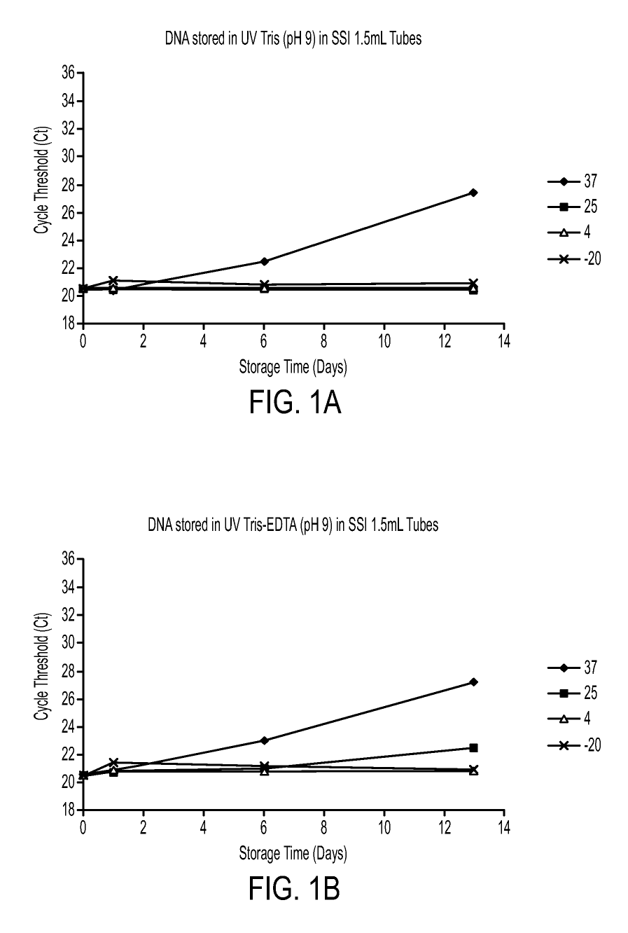

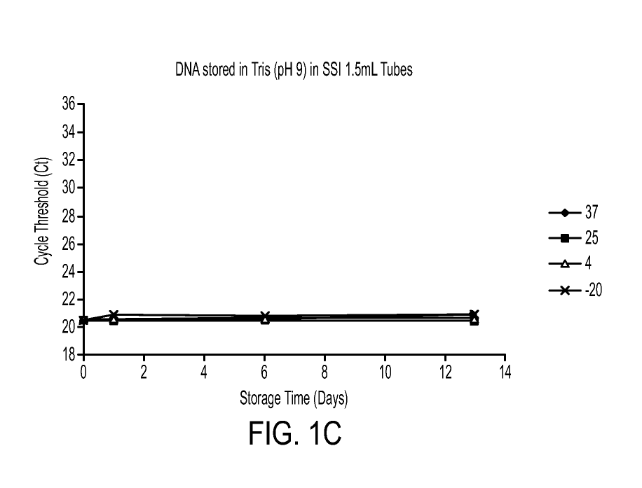

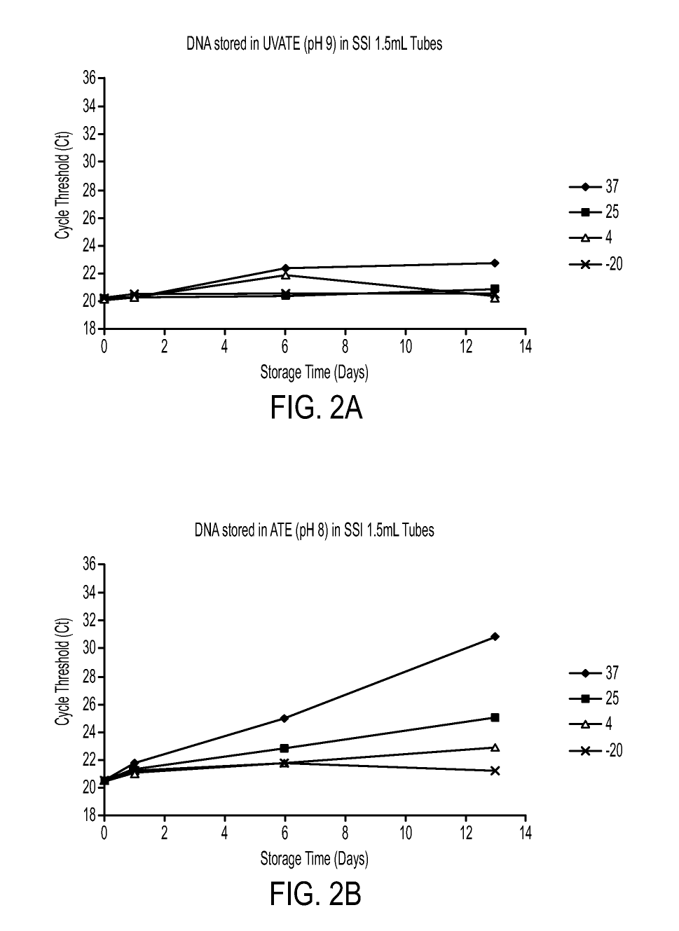

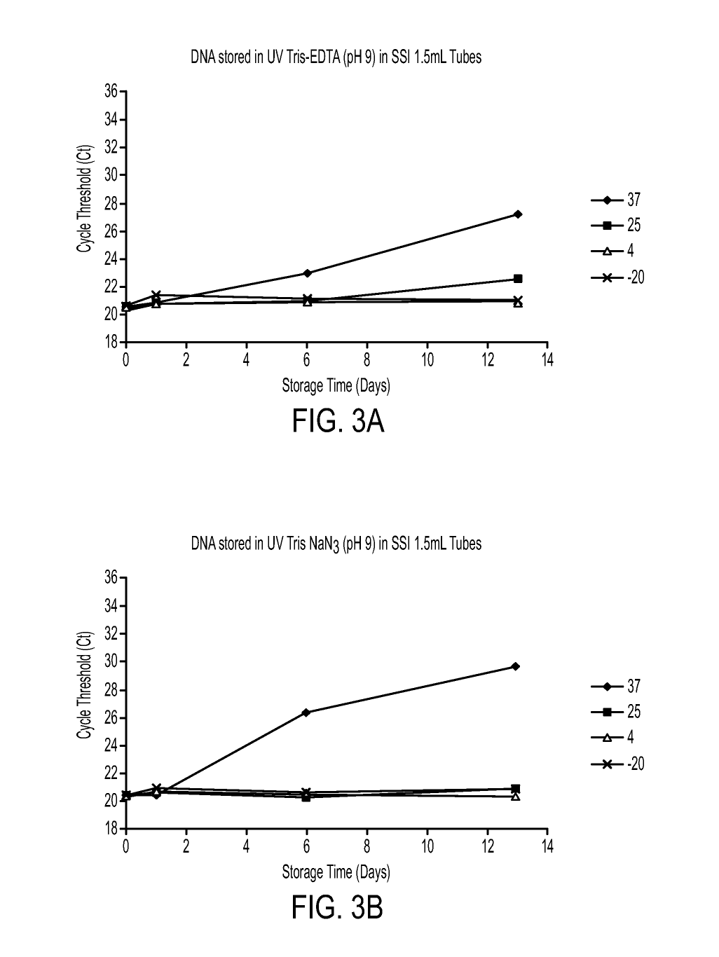

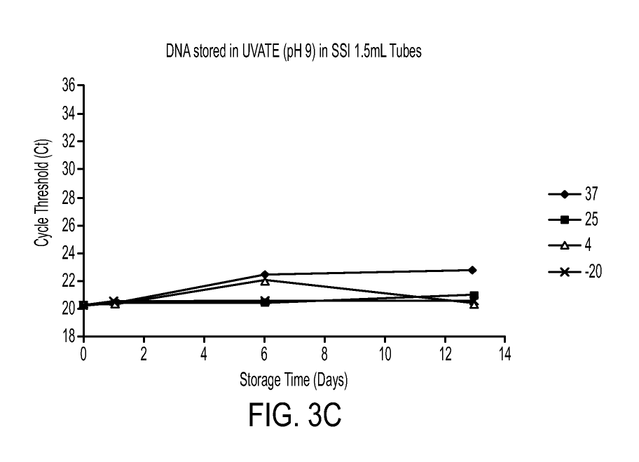

and stored for various amounts of time (1, 6, or 13 days) at different temperatures (−20° C., 4° C., 25° C., or 37° C.). As shown in FIG. 1, storage of DNA in UV-treated Tris (FIG. 1a) or Tris-EDTA (FIG. 1b) reduced the efficiency of PCR compared to storage of DNA in non-UV treated Tris (FIG. 1c) as demonstrated by the rise in cycle threshold over time in a real-time PCR reactions. UV treated Tris, EDTA, NaAzide (UVATE) buffer did not display the same reduction in PCR efficiency (FIG. 2a) as UV-treated Tris or Tris EDTA. Surprisingly, ATE buffer that was not UV-treated displayed even further reduced efficiency of PCR (FIG. 2b) than UV-treated Tris or Tris EDTA. It was found that both EDTA and NaAzide needed to be present in the Tris buffer prior to UV treatment in order to prevent a reduction of PCR efficiency, as Tris EDTA and Tris NaAzide did not convey the same benefit as UVATE (FIG. 3).

These results demonstrate that UVATE is a desirable buffer for storage of nucleic acids because it did not strongly negatively impact PCR as was observed with UV-Tris, UV-Tris EDTA, UV-Tris Sodium Azide or ATE. It is theorized that the benefit of UVATE is due to inhibition of the release of leachates present in SSI tubes (releasing agents and/or biocides) into the buffer or inhibition of the activity of leachates present in the buffer. These leachates are known as assay-interfering compounds.

### Example 3

### Comparison of ATE and UVATE in Multiple Types of Plastic Containers

### Material and Methods

E. coli or S. pneumoniae DNA was prepared as described in Example 1 and stored in each of the following types of tubes in either ATE or UVATE:

1) 200 microliter dome-top PCR tube

2) 1.5 mL SSI tube

3) 1.5 mL, 2 mL, or 50 mL Sarstedt tube

4) 15 mL BD Falcon tube

DNA was stored for 3, 7, or 14 days at −80° C., −20° C., 4° C., 25° C., or 37° C. Real-time PCR analysis was carried out as described in Example 1.

### Results

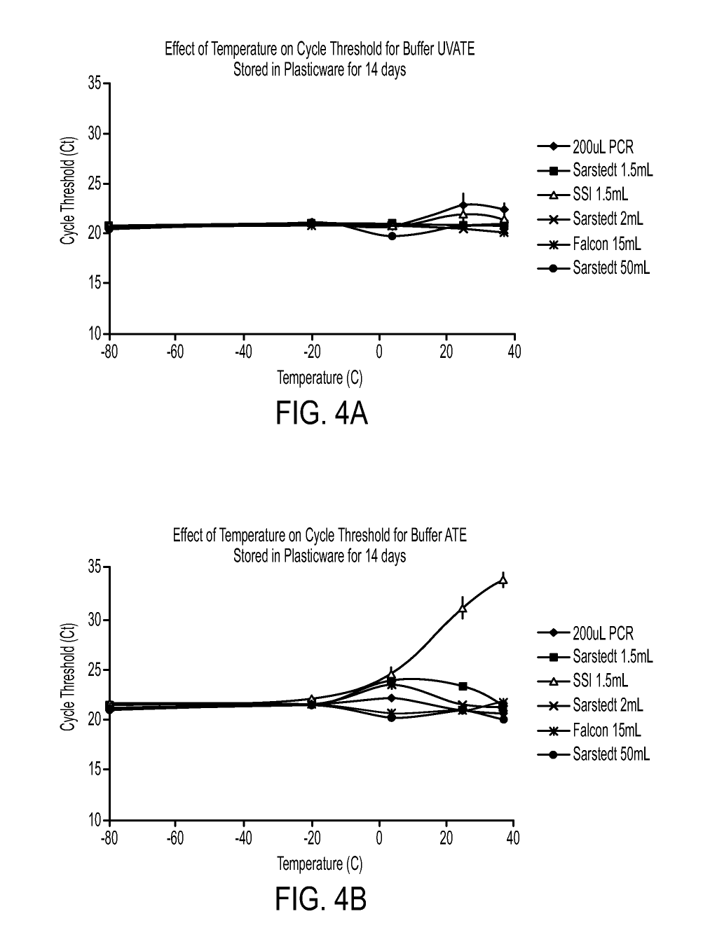

Example 1 demonstrated that UVATE was found to be a desirable storage buffer in SSI tubes, which are known to contain leachates that are detrimental to PCR and other assays. To further explore the properties of UVATE, bacterial DNA storage assays were performed in several different tube types: SSI tubes, Sarstedt tubes, Falcon tubes, and 200 microliter dome-top PCR tubes. FIG. 4 shows that even in Sarstedt and Falcon tubes, which are thought to contain fewer leachates than SSI tubes, storage of DNA in UVATE resulted in less inhibition of PCR. Thus, UVATE is a generally desirable storage buffer for storage of nucleic acids in plastic containers.

### Example 4

### Further Analysis of Different Buffers in Multiple Types of Plastic Containers

The process of exposing with ultraviolet light a reagent intended to subsequently contain and/or contact a nucleic acid is helpful when contaminating nucleic acid needs to be removed for diagnostic or research investigations.

Leachates and extractables derived from plasticware have been demonstrated in our work to inhibit PCR. Releasing agents such as erucamide and steramide plus biocides such as quaternary ammonium compounds have been described in the literature as assay interfering compounds which are derived from the disposable plasticware used to perform experiments and tests.

It is useful to have a UV decontaminated reagent intended to contain and/or contact a sample nucleic acid where the reagent also contains EDTA. It is additionally useful for the reagent to resist inhibition in downstream PCR due to leachates which may be derived from the plasticware. By adding NaN₃ prior to UV exposure, the composition will provide the above described benefits while allowing maximal PCR performance.

In order to carry out testing of nucleic acid storage reagent formulations and impact of UV treatments, S. pneumoniae and E. Coli DNA positive control DNA was prepared by diluting the applicable standard to 0.01 ng/μL using 10 mM Tris pH 8.5 and storing at −80° C. in 200 μl aliquots using domed-top PCR tubes. Separate aliquots were used for each time point to avoid DNA degradation arising from freeze/thaw cycles.

Different types of plasticware were assayed to determine whether they had any impact on cycle threshold (CO value for the S. pneumoniae assay when using either ATE (Tris, EDTA, and NaN₃) buffer or ultraviolet-treated ATE buffer (UVATE). 2 μL of S. pneumoniae DNA was seeded into 2 mL of either ATE or UVATE buffer to a final concentration of 0.01 ng/μL. Each fresh solution was amplified immediately to obtain a baseline Cₜ value. 50 μl aliquots were dispensed into 15 of each of the plasticware types (Table 1) for both buffers. Three of each type of plasticware were stored at 37° C., room temperature (25° C.), 4° C., −20° C. and −80° C. At each time point (3, 7 and 14 days) one 50 μl aliquot of each type of plasticware was removed and amplified using real-time PCR.

The UVATE and ATE buffers were assayed to specifically determine which components had any impact on Cₜ values for the Escherichia coli assay. Each buffer was prepared and UV-treated where necessary for 4 hours, and stored in 50 mL Sarstedt tubes. The Sarstedt 50 mL tubes were first washed by incubating Sigma water for 1 hour at room temperature, discarding the water and repeating the rinse with UV-treated Sigma water, and finally rinsed with a small amount of the buffer to be stored in them.

DNA-seeded buffers were prepared in 15 mL Falcon (Becton Dickinson) tubes, which had been pre-rinsed with the applicable buffer. 2 mL of buffer was added to which 2 μL of E. coli DNA was seeded and mixed by vortexing, giving a final concentration of 0.01 ng/μL. Each fresh solution was amplified immediately to obtain a baseline Cₜ value. 50 μl aliquots of each seeded buffer were dispensed into 12×1.5 mL Sarstedt O-ring screw top tubes and 12 SSI 1.5 mL tubes. Three of each type of plasticware were stored at 37° C., room temperature (25° C.), 4° C. and −20° C. At each time point (1, 6 and 13 days) one 50 μl aliquot of each type of buffer/tube was removed and amplified using real-time PCR. ATE pH 8.0=A, UVATE (Tris, EDTA and NaN₃) pH 9]=UA, Tris pH 9=T, UV Tris pH 9=UT, UV Tris EDTA pH 9=UTE, UV Tris and NaN₃ pH 9=at 0.04% UTN. Tris was at 10 mM, EDTA at 0.1 mM and NaN₃ at 0.04%.

A single mastermix was assembled according to the following concentrations: 0.5 (S. pneumoniae) or 0.625 (E. coli) units per reaction of PLATINUM Taq polymerase (Invitrogen), 1×PCR buffer (Invitrogen), 0.2 mM for each dNTP (Invitrogen), 4.5 mM (S. pneumoniae) or 5.5 mM (E. coli) MgCl₂ (Invitrogen), 0.01% v/v bovine serum albumin (BSA) (Sigma) and 6.5 μM propidium monoazide (PMA) (Biotium). PMA was cross-linked with any contaminating DNA by exposure to a light source for a total of 20 min, in alternative light/dark cycles of 30 sec and 60 sec respectively. After PMA treatment 0.2 μM of each of the forward/reverse primer and FAM-labeled probe was added. The mastermix was stored in 200 μl aliquots at −80° C. until required, ensuring every time point had an identical number of freeze/thaw cycles.

At each time point mastermix was dispensed into a Genedisc-72 ring using a liquid handling robot (QIAgility, QIAGEN). 10 μL of each of the DNA samples, positive control and negative control (UVATE or ATE) were inoculated in triplicate by hand into the Genedisc-72 ring, giving a final volume of 25 μL per reaction (10 μL sample and 15 μL mastermix). The ring was sealed using thermal sealing film.

Amplification was performed on a Rotor-Gene Q Thermal Cycler (QIAGEN) using the following reaction profile; 94° C. for 2 min for activation followed by 50 cycles of: 94° C. for 15 sec denaturation, 60° C. for 30 sec, and 72° C. for 20 sec for extension. Gains were set automatically before the first fluorescence acquisition using the default settings. Fluorescence acquisition to the green channel was performed after extension. Analysis was performed using a cycle threshold (Cₜ)=0.05 and Slope Correction=ON.

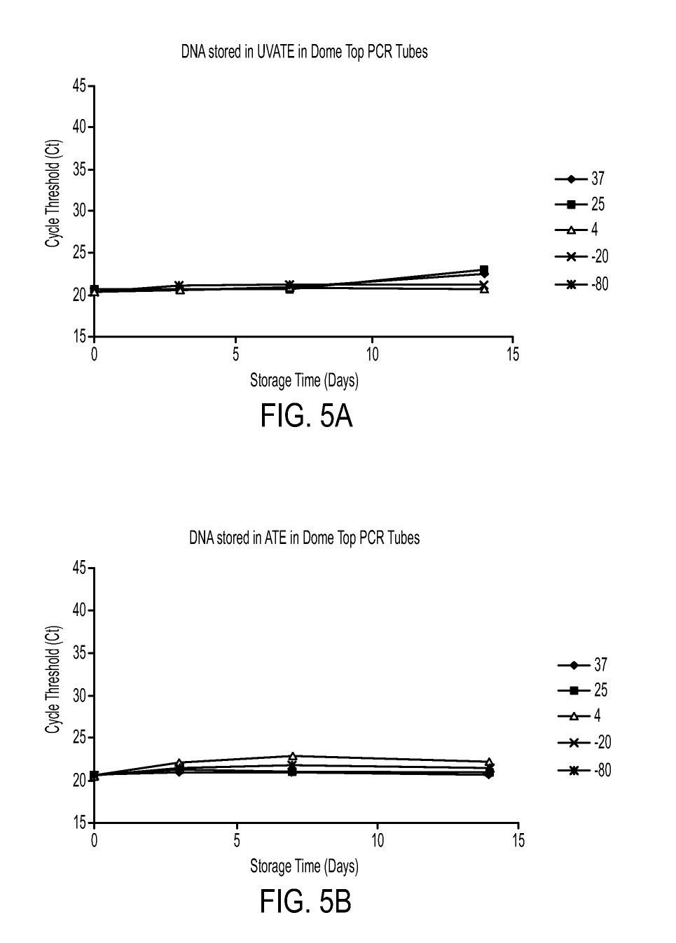

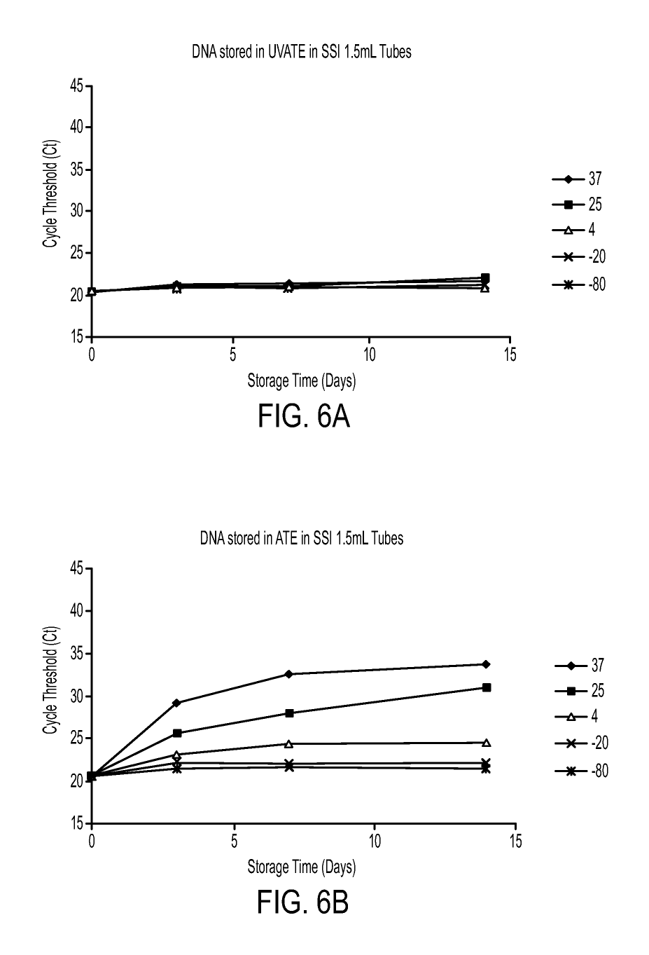

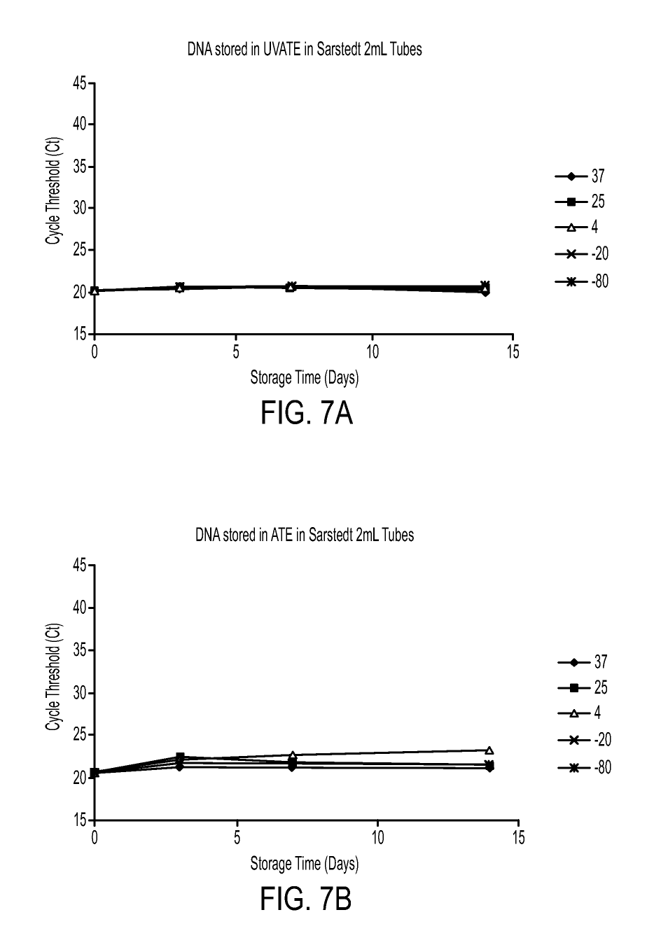

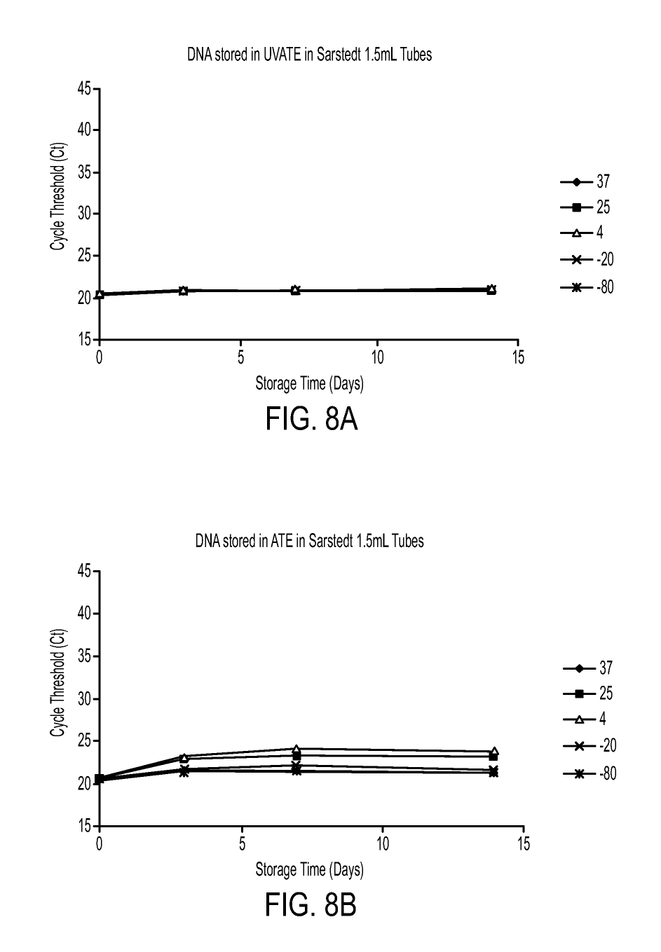

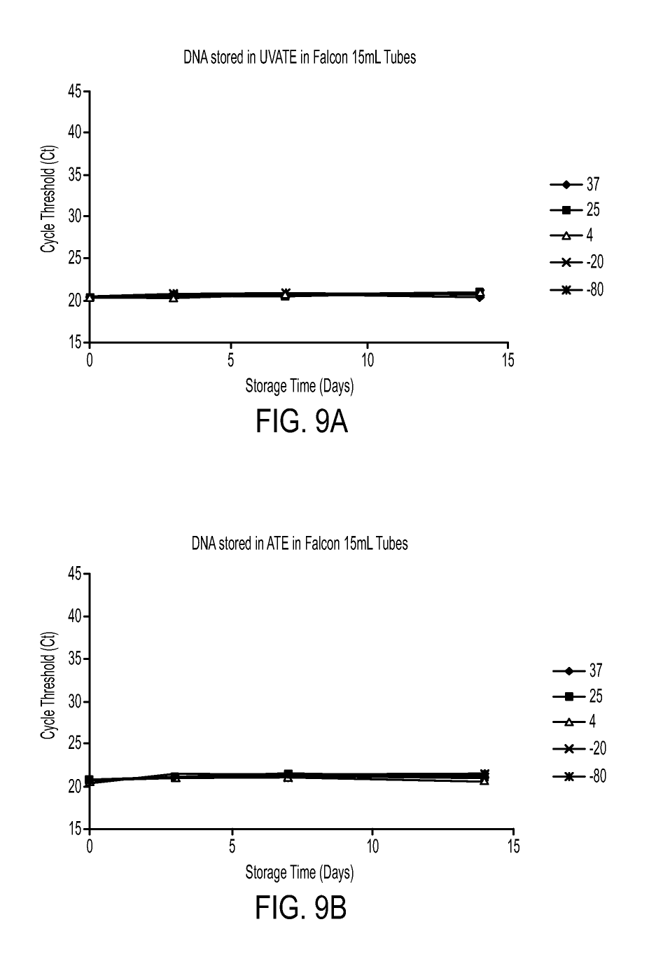

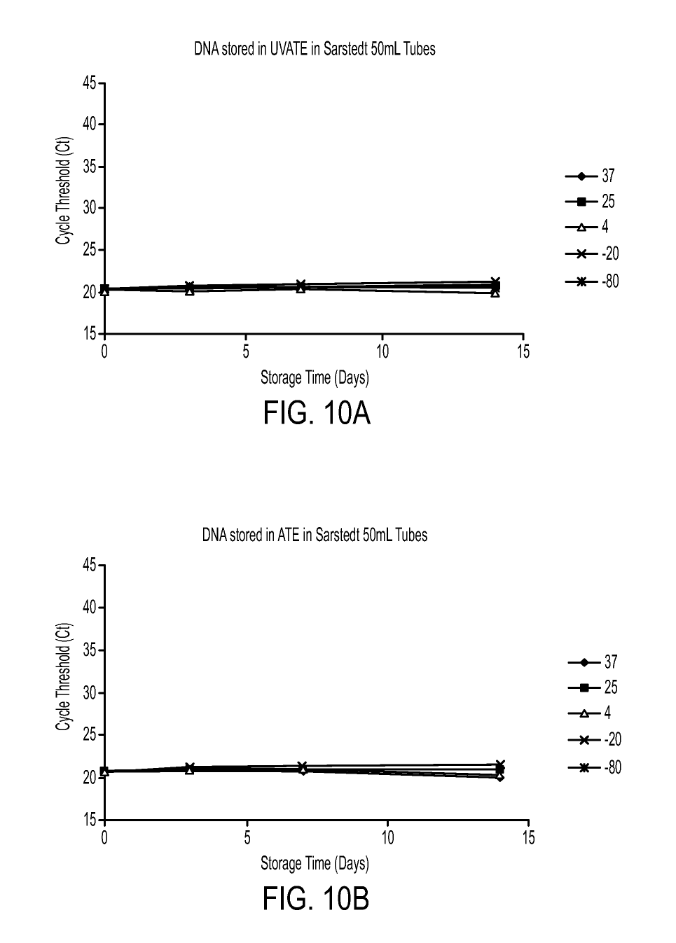

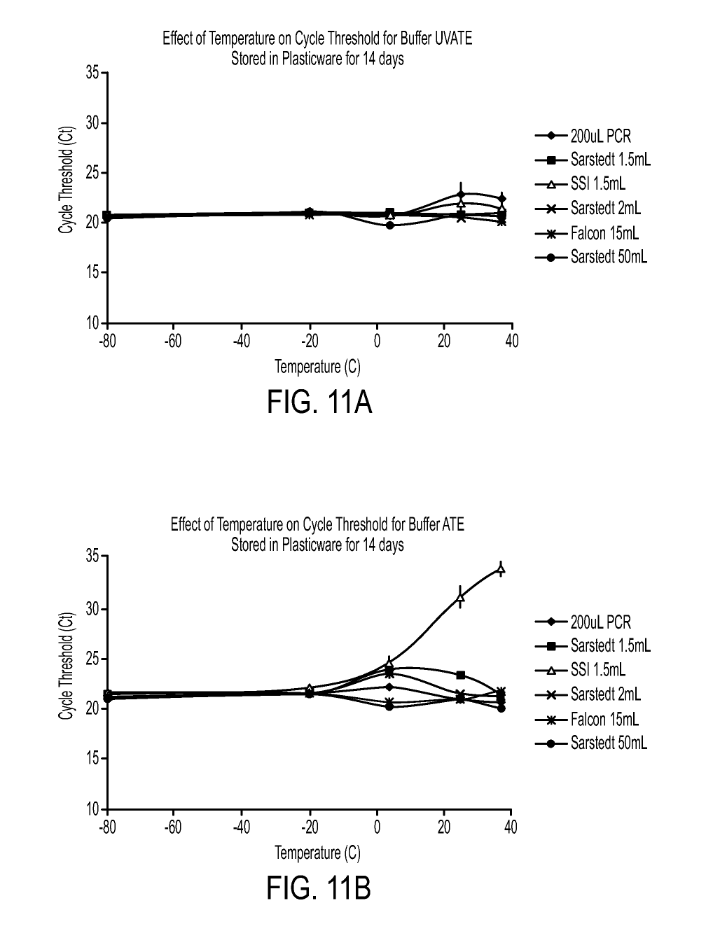

Both untreated ATE buffer and ultraviolet-treated ATE buffer (UVATE) were assessed as DNA storage mediums within a variety of plasticware. The change in Cₜ over 14 days at a variety of temperatures is detailed for dome top PCR tubes (FIG. 5), SSI 1.5 mL tubes (FIG. 6), Sarstedt 2 ml tubes (FIG. 7), Sarstedt 1.5 ml tubes (FIG. 8), BD Falcon 15 ml tubes (FIG. 9) and Sarstedt 50 ml tubes (FIG. 10). Aggregated data showing the effect of temperature on Cₜ value for each type of plasticware is presented in FIG. 11.

FIGS. 5-11 show that the decrease in PCR efficiency performed with nucleic acids stored in ATE varied depending on the type of tube tested, e.g., 15 mL Falcon tubes and 50 mL Sarstedt tubes. Falcon and Sarstedt tubes are thought to contain fewer leachates than, e.g., SSI tubes, so the PCR efficiency generally (regardless of the buffer used) may be partially linked to the type of tube used or the company that produces the tube. However, FIGS. 5-11 do show that PCR performance was generally improved by storing the nucleic acid in UVATE. This demonstrates that UVATE is generally useful as a storage buffer for optimized PCR performance.

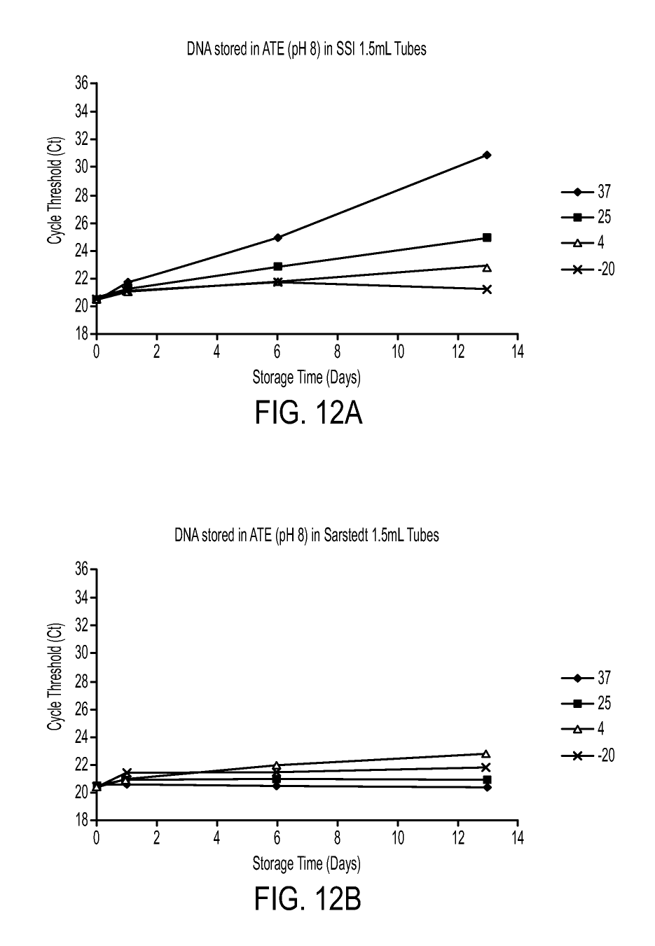

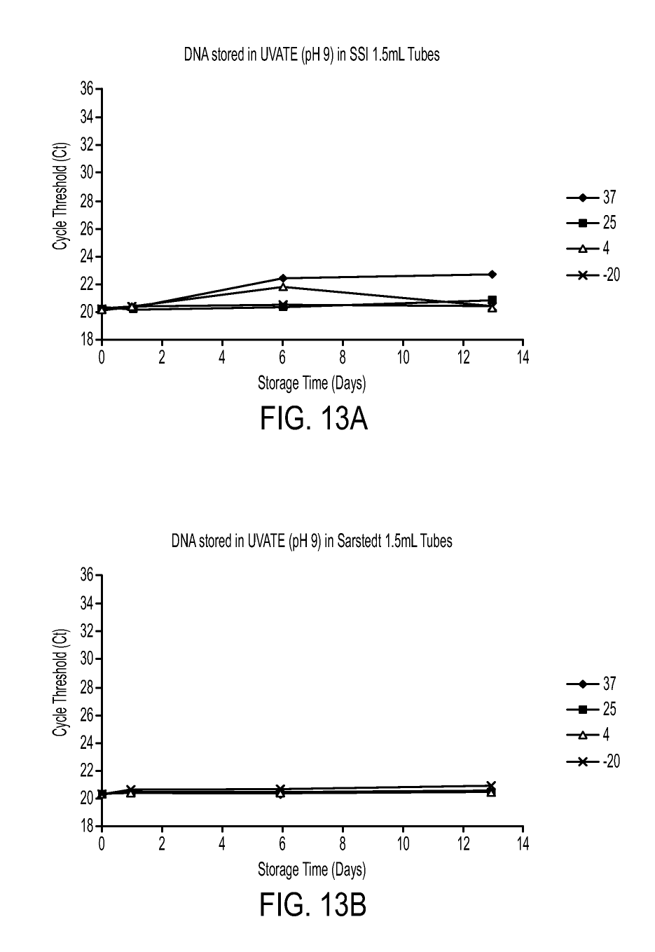

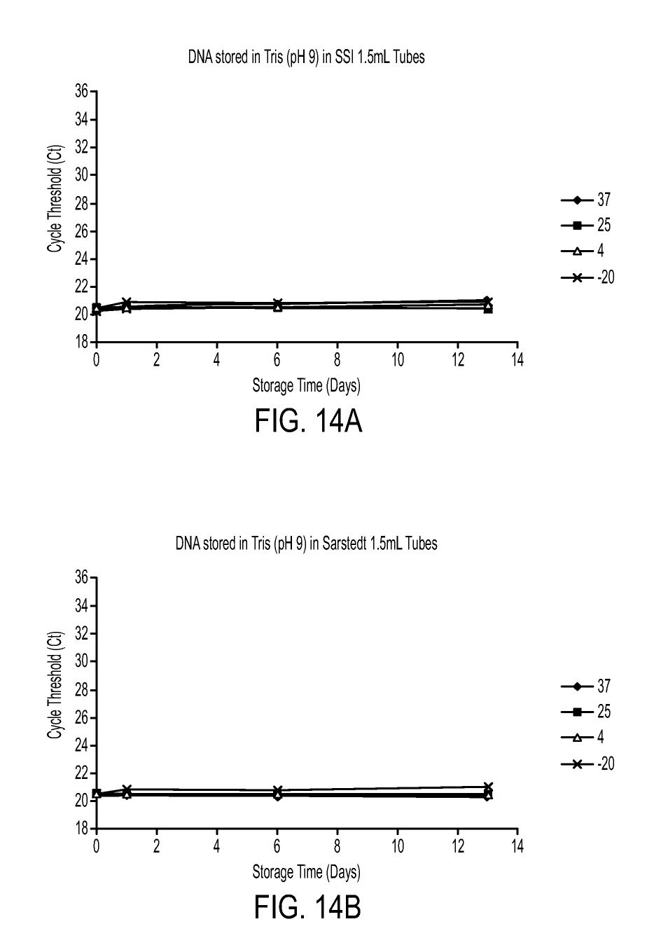

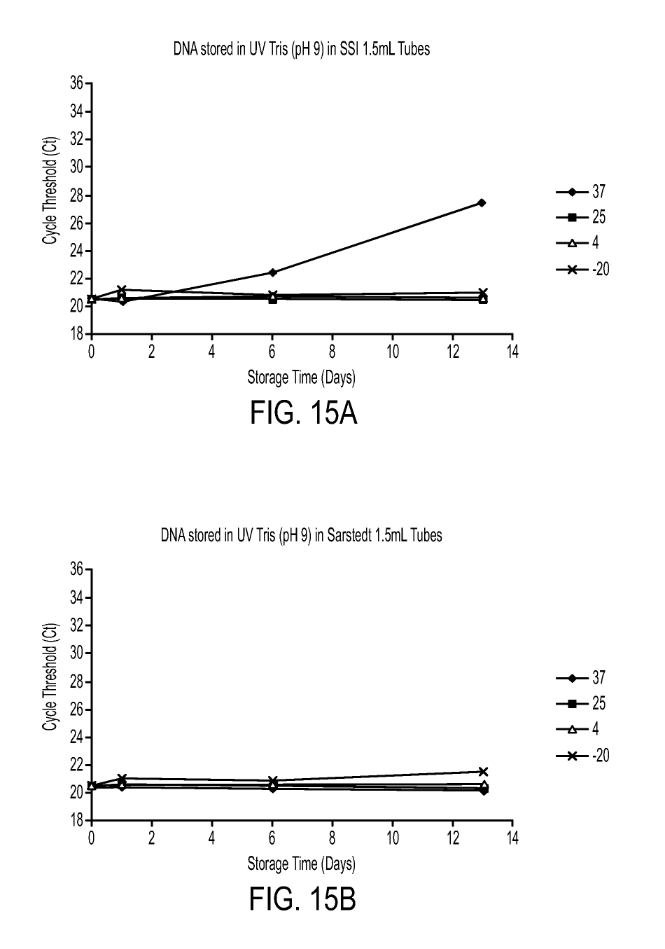

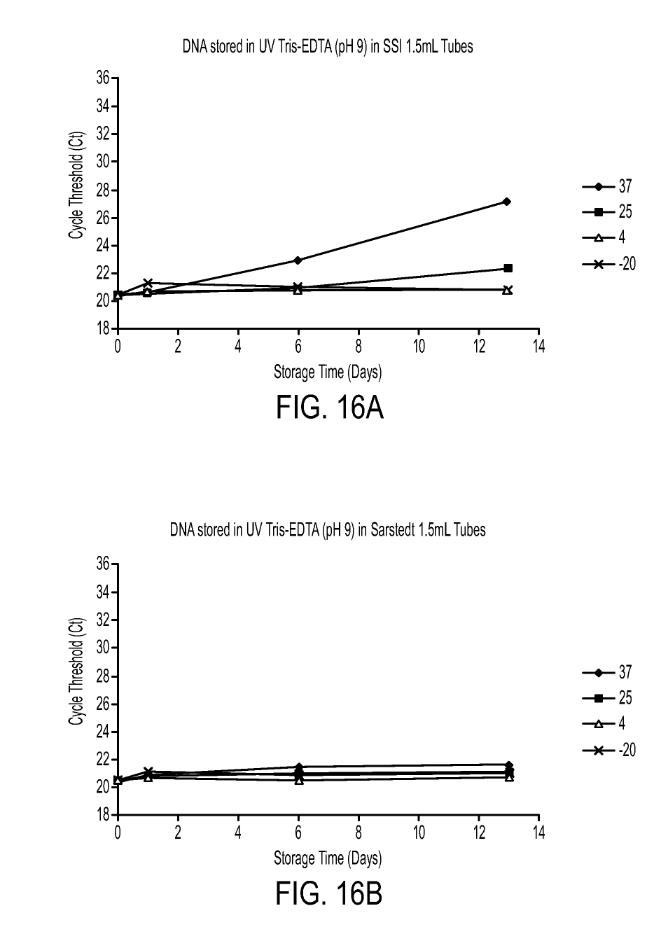

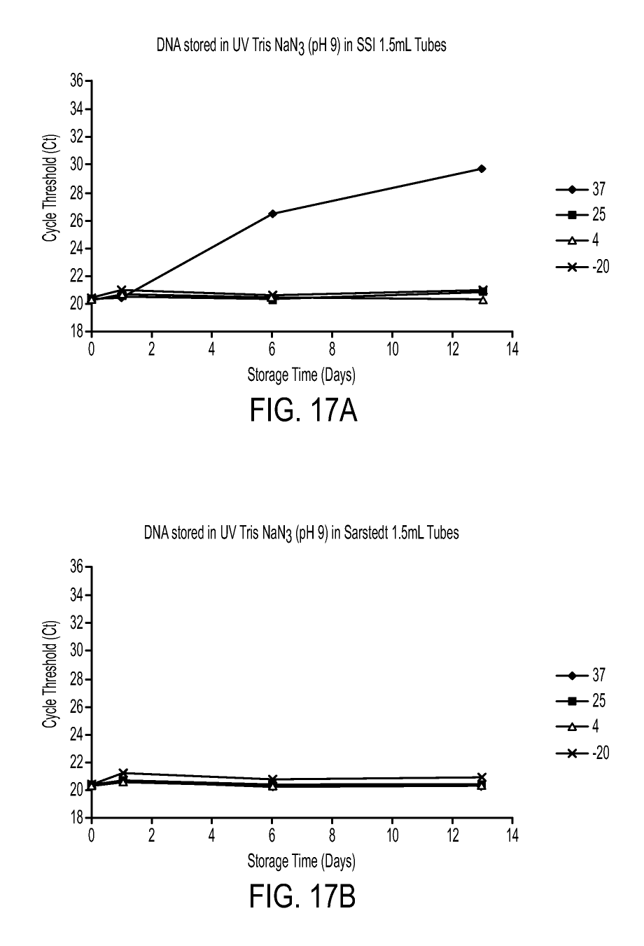

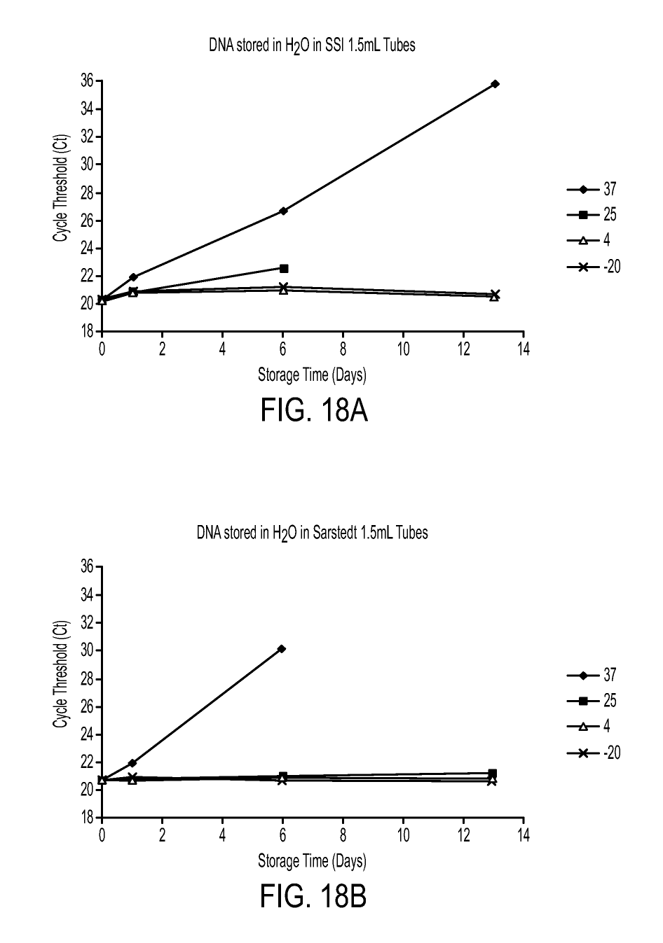

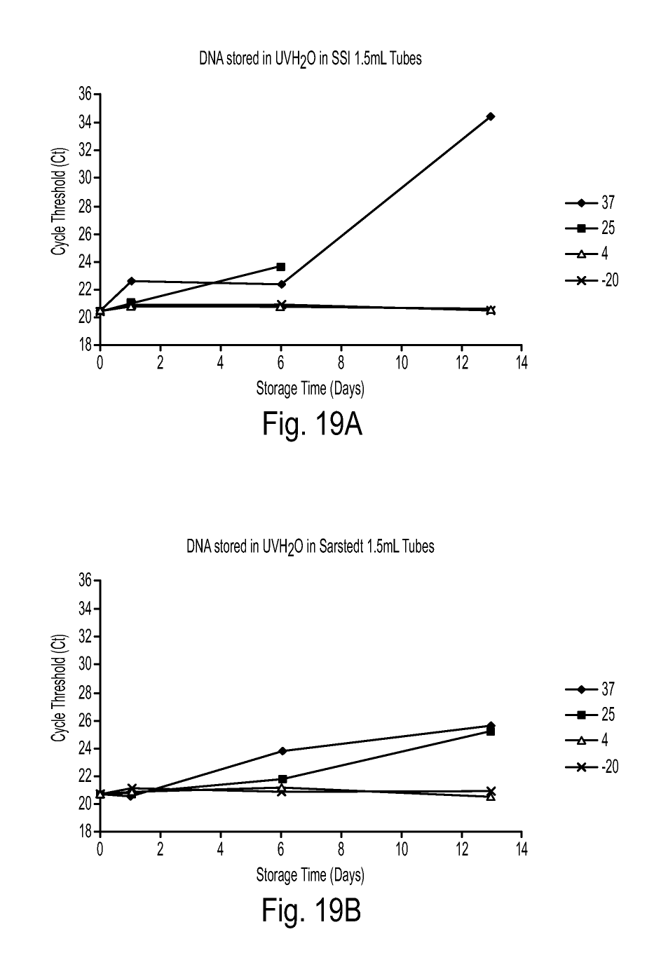

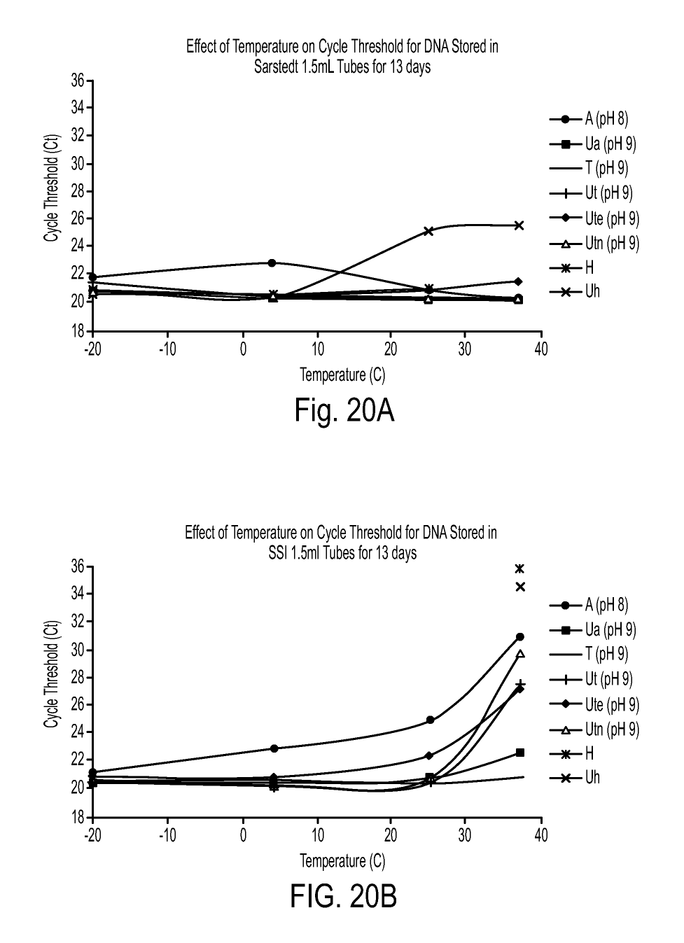

The components of ATE buffer were examined to determine their individual effects on the integrity of stored DNA. The change in Cₜ over a 13 day period at a variety of temperatures is detailed for buffer A (Tris, EDTA, and Azide, FIG. 12), buffer UA (UV-treated Tris, EDTA, and Azide, FIG. 13), buffer T (Tris, FIG. 14), buffer UT (UV-treated Tris, FIG. 15), buffer UTE (UV-treated Tris EDTA FIG. 16), buffer UTN (UV-treated Tris Azide, FIG. 17), buffer H (water, FIG. 18) and buffer UH (UV-Treated water, FIG. 19). Aggregated data showing the effect of temperature on Cₜ value for each of the buffers is presented in FIG. 20. FIGS. 12-20 show that only storage in Tris (not UV-treated) or UV-treated Tris, EDTA, Azide (UVATE) does not result in a decrease in PCR efficiency in both Sarstedt tubes and SSI tubes.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, e.g., as set forth in the United States Patent Office Manual of Patent Examining Procedures, Section 2111.03.

What is claimed is:

1. A composition, comprising Tris, ethylenediaminetetraacetic acid (EDTA), and sodium azide (NaN₃), wherein the composition has been previously exposed to an artificial source of ultraviolet irradiation.

2. The composition of claim 1, wherein the composition is a liquid.

3. The composition of claim 1, wherein the composition is dry.

4. The composition of claim 1, wherein the composition is lyophilized.

5. The composition of claim 1, wherein the composition is a solution and wherein Tris is present in an amount of 1.0 mM to 50 mM, EDTA is present in an amount of 0.05 mM to 2.0 mM, and NaN₃ is present in an amount of 0.01 to 0.3%.

6. The composition of claim 1, wherein the exposure to ultraviolet irradiation is for at least 15 minutes.

7. The composition of claim 6, wherein the exposure to ultraviolet irradiation is for 1 to 4 hours.

8. The composition of claim 1, wherein the ultraviolet irradiation is at a wavelength range from 250 to 320 nm.

9. A container, comprising:
(a) a composition comprising Tris, EDTA, and NaN₃, wherein the composition has been previously exposed to an artificial source of ultraviolet irradiation; and
(b) a nucleic acid.

10. The container of claim 9, wherein (a) and (b) are frozen.

11. The container of claim 9, wherein the container is plastic.

12. The container of claim 11, wherein the plastic comprises a polypropylene, a polystyrene, a polycarbonate, a cyclo-olefin, or a mixture thereof.

13. The container of claim 9, wherein the nucleic acid has not been previously exposed to an artificial source of ultraviolet irradiation.

14. The container of claim 9, wherein the nucleic acid is a DNA or cDNA.

15. The container of claim 9, wherein (a) and (b) are in a solution and wherein Tris is present in an amount of 1.0 mM to 50 mM, EDTA is present in an amount of 0.05 mM to 2.0 mM, and NaN3 is present in an amount of 0.01 to 0.3%.

16. The container of claim 9, wherein the exposure to ultraviolet irradiation is for at least 15 minutes.

17. The container of claim 16, wherein the exposure to ultraviolet irradiation is for 1 to 4 hours.

18. The container of claim 9, wherein the ultraviolet irradiation is at a wavelength range from 250 to 320 nm.

19. A method of producing a reagent, the method comprising:
(a) combining Tris, EDTA, and NaN3 in a solution; and
(b) exposing the solution to an artificial source of ultraviolet irradiation, thereby producing a reagent.

20. The method of claim 19, wherein the method further comprises drying the reagent.

21. The method of claim 19, wherein the method further comprises lyophilizing the reagent.

22. The method of claim 19, wherein Tris is present in an amount of 1.0 mM to 50 mM, EDTA is present in an amount of 0.05 mM to 2.0 mM, and NaN3 is present in an amount of 0.01 to 0.3%.

23. The method of claim 19, wherein the exposure to ultraviolet irradiation is for at least 15 minutes.

24. The method of claim 23, wherein the exposure to ultraviolet irradiation is for 1 to 4 hours.

25. The method of claim 19, wherein the ultraviolet irradiation is at a wavelength range from 250 to 320 nm.

26. A product obtainable by the method of claim 19.

27. A method, comprising performing a polymerase chain reaction (PCR) in the presence of a composition comprising Tris, EDTA, and sodium azide, wherein the composition has been previously exposed to an artificial source of ultraviolet irradiation.

28. The method of claim 27, wherein the PCR is real-time PCR.

29. The method of claim 27, wherein Tris is present in an amount of 1.0 mM to 50 mM, EDTA is present in an amount of 0.05 mM to 2.0 mM, and NaN3 is present in an amount of 0.01 to 0.3%.

30. The method of claim 29, wherein the exposure to ultraviolet irradiation is for at least 15 minutes.

31. The method of claim 30, wherein the exposure to ultraviolet irradiation is for 1 to 4 hours.

32. The method of claim 31, wherein the ultraviolet irradiation is at a wavelength range from 250 to 320 nm.
